(19) United States
(12) Patent Application Publication
(10) Pub. No.: US 2020/0265540 A1
(43) Pub. Date: Aug. 20, 2020

(54) System, Method and Device for Registration and Identification of Prescribed and Legally Dispensed Cannabis Products

(71) Applicant: Everett Ogden, Ponte Vedra Beach, FL (US)

(72) Inventor: Everett Ogden, Ponte Vedra Beach, FL (US)

(21) Appl. No.: 16/791,492

(22) Filed: Feb. 14, 2020

Related U.S. Application Data
(60) Provisional application No. 62/806,279, filed on Feb. 15, 2019.
(60) Provisional application No. 62/811,935, filed on Feb. 28, 2019.
(60) Provisional application No. 62/848,366, filed on May 15, 2019.

(51) Int. Cl. G06Q 50/26; G06Q 30/00; G16H 70/40; G16H 10/60; G06K 7/10; H04W 4/20

(52) U.S. Cl. CPC G06Q 50/265; G06Q 30/0185; H04W 4/20; G16H 10/60; G06K 7/10366; G16H 70/40

(57) ABSTRACT

A system, method and device that allows medically prescribed cannabis products, such as individual cigarettes, e-cigarettes or cannisters, to be tracked, registered, identified and matched with both legal prescriptions and the patients themselves, such that law enforcement may readily determine if an individual is legally in possession of a medically prescribed cannabis product.

### BACKGROUND OF THE INVENTION

This invention relates generally to the field of cannabis and more particularly to medically prescribed and legally dispensed cannabis containing products.

Cannabis, which comes in the form of marijuana (dried flower buds), resin (hashish) or oil (hashish oil), is known to address, treat or relieve symptoms of various diseases and conditions, to reduce nausea and vomiting during chemotherapy, to improve appetite in HIV/AIDS patients, and to treat chronic pain. Cannabis is also extensively used as a recreational drug.

A significant number of states have enacted legislation allowing the controlled manufacture, sale and use of recreational cannabis products or medically prescribed cannabis products, whether in the form of marijuana as plant material, resins, oils or extracts in natural form or as an included cannabis product in cigarettes or e-cigarettes, also known as e-vaporizers. The recreational cannabis products are dispensed directly from retail establishments, while the medically prescribed cannabis products are prescribed by licensed medical personnel and dispensed by licensed manufacturers and/or directly by licensed medical personnel.

A major issue of concern in both the states that have enacted such legislation and in those states that have not is the current inability to monitor and track the cannabis products through the growth, manufacturing, distribution and possession segments of the commercial transactions. This issue is of extreme concern relative to the medically prescribed cannabis products after they have been distributed to the end-users or patients, as it is thought that a significant amount of the legally prescribed cannabis may then be illegally resold by those possessing legal prescriptions to individuals, including minors, lacking the necessary medical prescriptions.

Unlike recreational use cannabis cigarettes, cannabis cigarettes produced for distribution based on legal medical prescriptions are produced with filters. E-cigarettes for medicinal prescriptions may be produced in single dose forms or in a reusable form in conjunction with disposable individual tanks, vials or cannisters. It is an object of this invention to provide a method, system and device for medically prescribed cannabis products, and in particular medically prescribed cannabis cigarettes and e-cigarettes that allow individual cannabis products to be tracked, identified and matched with both legal prescriptions and the patients themselves, such that law enforcement may readily determine if an individual in possession of a cannabis product is legally in possession of a medically prescribed cannabis product.

Although primarily described herein with regard to medically prescribed cannabis, the system, method and devices may also be utilized in states allowing recreational cannabis use where laws require the recreational cannabis to be purchased from state-licensed retail outlets.

### SUMMARY OF THE INVENTION

In brief summary, the invention in various embodiments is a system, method and device that allows individual cannabis containing products (referred to herein collectively as “cannabis products”), and in particular medically prescribed cannabis cigarettes, e-cigarettes or cannisters (referred to herein collectively as “medically prescribed cannabis products”) to be tracked, identified and matched with both legal prescriptions and the patients themselves, such that law enforcement may readily determine if an individual in possession of a medically prescribed cannabis product is legally in possession. Identification information for tracking purposes is physically connected or associated to the medically prescribed cannabis products, such as being imprinted or otherwise attached to or on the filter of each cannabis cigarette, on the e-cigarette device, or on the e-cigarette cannisters. Alternatively, an electronic tagging device, such as a miniaturized radio frequency identification (RFID) chip, may be embedded in or attached to the medically prescribed cannabis products. Additionally, genetic encoding or application of bioluminescent organisms or fluorescent proteins directly or indirectly (such as through fertilization) into the marijuana in various growth stages (seed to plant) or into or onto filters or cigarette papers may be utilized for tracking purposes. These marking methods and means are referred to herein as “tracking identifiers (TID)”. A database is maintained such that each TID is associated with information applying to each medically prescribed cannabis product which identifies the individual in legal possession of the cannabis, i.e., individual to whom the cannabis has been legally prescribed. The TID may further provide information as to the licensed manufacturer, the licensed prescriber, and/or the precise contents. The TID therefore provides law enforcement with a fast means to determine legal possession of the medically prescribed cannabis product.

Software-based tracking systems incorporating scanners or other recognitions means are thereby able to match TID information from any medically prescribed cannabis product to law enforcement, government or independent databases. Likewise, wireless Internet communications may be utilized to match TID information.

The system and method comprise the establishment of a cannabis product database containing information relevant to the growing, manufacture of cannabis products, transportation, inventory, sale and distribution of cannabis products and in particular medically prescribed cannabis products. The cannabis product database further contains information as to individual TID's associated with each medically prescribed cannabis product, information as to the medical personnel prescribing each medically prescribed cannabis product, information as to the legally licensed dispensing entity dispensing each medically prescribed cannabis product, and information as to the individual to which each medically prescribed cannabis product is prescribed and dispensed. The system and method further comprise means and method of physically connecting or associating TID information to each medically prescribed cannabis product. The system and method further comprise means and method enabling law enforcement personnel to extract the TID information from each medically prescribed cannabis product, such as visually, with an optical scanner, RFID reader or the like, communicate the information to the database for matching purposes, such as by direct communication from the optical scanner, RFID reader or the like, or by inputting the TID information into a portable computer and communicating via wireless Internet with the database, and means and method to receive confirmation from the database that the medically prescribed cannabis product is possessed by the appropriate individual.

### BRIEF DESCRIPTION OF THE DRAWINGS

FIG. 1 is an illustration of a cannabis cigarette having a TID imprinted on the filter.

FIG. 2 is an illustration of an e-cigarette having a TID imprinted thereon.

FIG. 3 is an illustration of an e-cigarette cannister having a TID imprinted thereon.

FIG. 4 is a flow chart showing a basic embodiment of the system and method of the invention.

### DETAILED DESCRIPTION OF THE INVENTION

In general, the invention in various embodiments is a system, method and device that allows individual cannabis containing products (referred to herein collectively as “cannabis products”), and in particular medically prescribed cannabis cigarettes 11, e-cigarettes 13 or cannisters 14 (referred to herein collectively as “medically prescribed cannabis products”) to be tracked, identified and matched with both legal prescriptions and the patients themselves, such that law enforcement may readily determine if an individual in possession of a medically prescribed cannabis product is legally in possession.

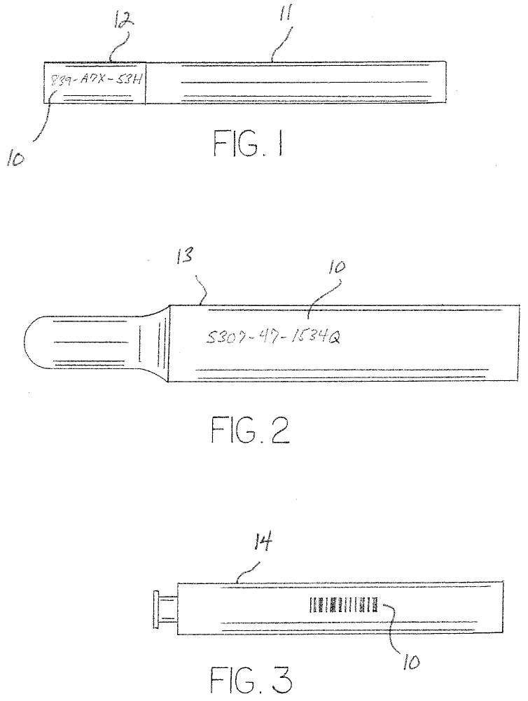

Identification information for tracking purposes is physically connected or associated to the medically prescribed cannabis products, such as being inscribed, stamped, imprinted, permanently labelled by adhesive label, or otherwise attached to or on the filter 12 of each cannabis cigarette 11, on the e-cigarette device 13, or on the e-cigarette cannisters 14, as shown in FIGS. 1-3. The identification information may even be inserted or hidden within the medically prescribed cannabis products. Alternatively, an electronic tagging device, such as a miniaturized radio frequency identification (RFID) chip, may be embedded in or attached to the medically prescribed cannabis products. Additionally, genetic encoding or application of bioluminescent organisms or fluorescent proteins directly or indirectly (such as through fertilization) into the marijuana in various growth stages (seed to plant) or into or onto filters or cigarette papers may be utilized for tracking purposes. These marking methods and means are referred to herein as “tracking identifiers (TID)” 10.

The TID 10 enables enabling law enforcement personnel to determine whether an individual is in legal possession of the medically prescribed cannabis product. Most preferably, the TID 10 contains information such as the licensed manufacturer, the licensed prescriber, the licensed dispenser, and/or the precise contents of the medically prescribed cannabis product. The TID 10 is physically associated with the medically prescribed cannabis product by the manufacturer or dispenser of the medically prescribed cannabis product, depending on the type of product being dispensed.

A cannabis database is established and maintained such that the TID 10 associated with each medically prescribed cannabis product is available to law enforcement personnel. This information is provided to the cannabis database by manufactures, distributors, prescribers and dispensers no later than the time the medically prescribed cannabis product is dispensed to the individual legally receiving the medically prescribed cannabis product.

In addition to this information, information is provided to the database by the prescribers and dispensers which identifies the individual to whom the cannabis has been legally prescribed, such as the individual's name, driver's license number, social security number, photograph or similar identifying information. In this manner the TID of the medically prescribed cannabis product dispensed to an individual is associated in the database with legally prescribed individual identification information.

Software-based tracking systems incorporating scanners or other recognitions means are provided to match TID 10 information from any medically prescribed cannabis product to law enforcement, government or independent databases. Likewise, wireless Internet communications may be utilized to match TID 10 information. For example, an optical scanner or RFID reader is utilized to extract the data from the TID 10, this data then being communicated to the database where it is matched with the information in the database as to the individual legally entitled to possess the medically prescribed cannabis product. In the event the individual possessor being detained and questioned is not the individual legally in possession of the medically prescribed cannabis product, appropriate action may be taken by law enforcement personnel.

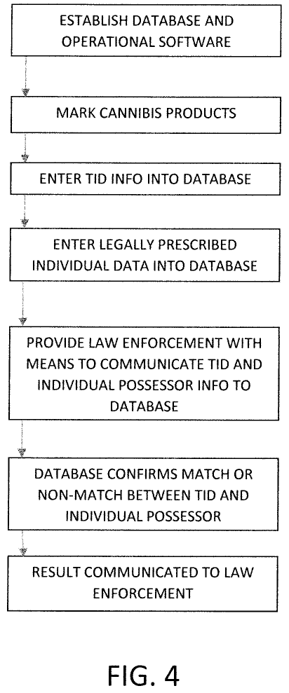

As illustrated in FIG. 4, the system and method comprise the establishment of a cannabis product database containing information relevant to the growing, manufacture of cannabis products, transportation, inventory, sale and distribution of cannabis products and in particular medically prescribed cannabis products. The cannabis product database further contains various amounts of information, such as information as to individual TID 10's associated with each medically prescribed cannabis product, information as to the medical personnel prescribing each medically prescribed cannabis product, information as to the legally licensed dispensing entity dispensing each medically prescribed cannabis product, and information as to the individual to which each medically prescribed cannabis product is prescribed and dispensed (i.e., legally prescribed individual identification information).

The system and method further comprise means and method of physically connecting or associating TID 10 information to each medically prescribed cannabis product. The system and method further comprise means and method enabling law enforcement personnel to extract the TID 10 information from each medically prescribed cannabis product, such as visually, with an optical scanner, RFID reader, cellphone camera, laptop camera, etc., communicate the information to the database for matching purposes, such as by direct communication from the optical scanner, RFID reader etc., or by inputting the TID 10 information into a portable computer and communicating via wireless Internet with the database, and means and method to receive confirmation from the database that the medically prescribed cannabis product is possessed by the appropriate individual.

The TID 10 may incorporate a direct reference to the legal possessor of the medically prescribed cannabis product, such as the individual's driver license or social security number. The TID 10 may be in alpha-numeric form or may be in the form of a machine-readable indicia, such as a bar or Q-code. The TID 10 may be applied by the manufacturer, by a governmental entity, or by the distributor (i.e., pharmacy or doctor's office). In addition to imprinting the TID 10, non-removable adhesive stickers or other articles may be utilized to affix the TID 10 to the medically prescribed cannabis product.

The TID 10 therefore provides law enforcement with a quick means to determine legal possession of the medically prescribed cannabis product. Upon stopping an individual in possession of a medically prescribed cannabis product, the law enforcement official may read the TID 10 visually or through electronic communication with the RFID chip and request confirmation identification if the TID 10 contains a personal identifier (e.g., driver license number), may enter the TID 10 into a communications device (e.g., laptop computer, cellphone) in communication with the TID 10 database, or may utilize a scanning device (e.g., a smartphone or bar code reader) to input the TID 10 directly to the database system. The law enforcement officer then communicates possessor individual identification information to the database.

The database software then determines whether the identification information of the possessing individual and the TID 10 of a legally prescribed cannabis product as communicated from the field by law enforcement personnel matches the legally prescribed individual identification data associated with the TID 10 data contained in the database. If there is a match, then the possessor is legally in possession of the legally prescribed cannabis product. If there is not a match, then the possessor is not legally in possession and further action may be taken.

Thus, the invention is a method of registration and identification of legally prescribed cannabis products, whereby law enforcement personnel may determine whether an individual is lawfully in possession of a legally prescribed cannabis product, comprising the steps of: establishing a database and operational software receiving and processing TID 10 information on legally prescribed cannabis products and identification information on legally prescribed individuals; marking said legally prescribed cannabis products with said TID 10 information; communicating to and entering said TID 10 information into said database; communicating to and entering legally prescribed individual identification information into said database; providing law enforcement personnel with equipment to read said TID 10 information on one of said legally prescribed cannabis products and to communicate said TID 10 information to said database; communicating said TID 10 information to said database; communicating possessor individual identification information to said database; said operational software forming a determination of whether said possessor individual identification information matches said legally prescribed individual identification information for said one of said legally prescribed cannabis products; and communicating said determination to said law enforcement personnel. Furthermore to this, the invention wherein said step of marking said legally prescribed cannabis products with said TID 10 information is performed by the manufacturer, the dispenser or the prescriber of said legally prescribed cannabis products; wherein said steps of communicating to and entering said TID 10 information into said database and communicating to and entering legally prescribed individual identification information into said database are performed at separate times or at the same time; wherein said step of providing law enforcement personnel with equipment to read said TID 10 information on one of said legally prescribed cannabis products and to communicate said TID 10 information to said database comprises providing an optical scanner, RFID reader, cellphone camera or laptop camera; wherein said step of communicating said TID 10 information to said database is performed by an optical scanner, RFID reader, cellphone or laptop computer; wherein said TID 10 information is chosen from the group of TID 10 information consisting of the licensed manufacturer, the licensed prescriber, the licensed dispenser, and the precise contents of said medically prescribed cannabis products and/or wherein said TID 10 information comprises said legally prescribed individual identification information.

Software-based tracking systems incorporating scanners or other recognitions means match TID 10 10 information from individual cannabis containing object to law enforcement databases. For example, software may operate in conjunction with a scanning registration number (bar code) on cannabis foods, drinks, cigarettes and other products containing cannabis. Tracking ownership of recreational and medical prescription cannabis with the current law enforcement driver's license scanners and national data base may be accomplished. Matching data will enforce state and federal legal possession laws. The software data can also be used to track cannabis products in order to identify inferior, counterfeit, or tainted cannabis foods, drinks, cigarettes, (CBD) oils.

Software data can be used by the IRS in order to implement taxation on cannabis products such as foods, drinks, cigarettes or extract based (CBD) oil products. Software may be used to complement other management and tracking systems used to track the registration of legal seed to sell state cannabis growers requirements.

Furthermore, it is contemplated that laws may be enacted to further prevent unauthorized distribution or resale of the medically prescribed cannabis products, such as for example laws to make possession of an altered legally prescribed product illegal, such as by removing or altering the TID 10.

It is understood that equivalents and substitutions to elements and/or steps described above may be obvious to those of skill in the art, and therefore the true scope and definition of the invention is to be as set forth in the following claims.

1. A method of registration and identification of legally prescribed cannabis products, whereby law enforcement personnel may determine whether an individual is lawfully in possession of a legally prescribed cannabis product, comprising the steps of:
establishing a database and operational software receiving and processing TID information on legally prescribed cannabis products and identification information on legally prescribed individuals;
marking said legally prescribed cannabis products with said TID information;
communicating to and entering said TID information into said database;
communicating to and entering legally prescribed individual identification information into said database;
providing law enforcement personnel with equipment to read said TID information on one of said legally prescribed cannabis products and to communicate said TID information to said database;
communicating said TID information to said database;
communicating possessor individual identification information to said database;
said operational software forming a determination of whether said possessor individual identification information matches said legally prescribed individual identification information for said one of said legally prescribed cannabis products; and
communicating said determination to said law enforcement personnel.

2. The method of claim 1, wherein said step of marking said legally prescribed cannabis products with said TID information is performed by the manufacturer of said legally prescribed cannabis products.

3. The method of claim 1, wherein said step of marking said legally prescribed cannabis products with said TID information is performed by the dispenser of said legally prescribed cannabis products.

4. The method of claim 1, wherein said step of marking said legally prescribed cannabis products with said TID information is performed by the prescriber of said legally prescribed cannabis products.

5. The method of claim 1, wherein said steps of communicating to and entering said TID information into said database and communicating to and entering legally prescribed individual identification information into said database are performed at separate times.

6. The method of claim 1, wherein said steps of communicating to and entering said TID information into said database and communicating to and entering legally prescribed individual identification information into said database are performed at the same time.

7. The method of claim 1, wherein said step of providing law enforcement personnel with equipment to read said TID information on one of said legally prescribed cannabis products and to communicate said TID information to said database comprises providing an optical scanner, RFID reader, cellphone camera or laptop camera.

8. The method of claim 1, wherein said step of communicating said TID information to said database is performed by an optical scanner, RFID reader, cellphone or laptop computer.

9. The method of claim 1, wherein said TID information is chosen from the group of TID information consisting of the licensed manufacturer, the licensed prescriber, the licensed dispenser, and the precise contents of said medically prescribed cannabis products.

10. The method of claim 1, wherein said TID information comprises said legally prescribed individual identification information.
